(19) United States
(12) Patent Application Publication
(10) Pub. No.: US 2007/0071799 A1
(43) Pub. Date: Mar. 29, 2007

(54) Anchor point for affixing tubes and the like to skin

(76) Inventor: Christopher Tacklind, Palo Alto, CA (US)

Correspondence Address: Chris Tacklind, 250 Cowper Street, Palo Alto, CA, 94301, US

(21) Appl. No.: 11/179,414

(22) Filed: Jul. 12, 2005

(51) Int. Cl. A61F 13/02

(52) U.S. Cl. 424/448

(57) ABSTRACT

An Anchor Point is presented for anchoring a tube or the like to the body. The anchors are long lasting and improve patient mobility. In one embodiment the Anchor Point consists of two identical self adhesive flat components. At the proximal end, each Anchor Point component has an interrupted patch that adheres to the skin. A tab is provided from the center of each patch. At the distal end, the tabs wrap around the tube to be anchored. The tabs stick to each other and the tube leaving no adhesive exposed. Assembly may be by the patient, a practitioner, or may be factory assembled. Tension on the tube is transferred through the tab to the patch center. The forces on the patch are then substantially in shear and so the tension does not peel the patch free.

There are no cross-reference or related applications.

This invention was not sponsored by any Federal R&D agencies.

No appendices are attached.

### BACKGROUND OF THE INVENTION

Various types of tubes must be attached to a human body for medical purposes. Such tubes include but are not limited to intravenous tubes, and catheter tubes. The tubes are often flexible, allowing them to move with the body. The insertion point or attachment point must be protected from external forces on the tube or the tube can be dislodged from the body. Similarly, instrumentation wires and other implements may be attached to the body. Devices such as insulin pumps may also be attached to the skin. Several techniques are in common use by health care professionals for securing such items using tape. In practice, none is effective for long, as the tape peels away from the skin. This substantially limits user mobility. Assemblies of snaps and other fastening devices have been devised but are cumbersome and expensive.

### SUMMARY OF THE INVENTION

It is therefore an object of the present invention to provide a point for attaching medical implements such as a tube, a wire, devices, and the like, to the body. This relieves forces on the insertion point and provides necessary compliance allowing for improved patient mobility. Further, the invention transfers the forces to the center of the Anchor Point thereby preventing peeling forces on the point, making the attachment long lasting. The size of the Tether Point may be selected to suit a wide range of applications. The Tether Point components are readily die cut from adhesive tape stock in mass production. Multiple release liners may be arranged to simplify attachment. A method is provided for applying a pair of components forming an Anchor Point. The components may be assembled onto the skin or may be factory assembled. This results in inexpensive, convenient, long lasting, and secure attachments to the body. A flat sterile paper pouch of Anchor Points will fit into any kit such as a catheter kit or an I. V. kit.

### BRIEF DESCRIPTION OF THE DRAWINGS

FIG. 1 is an oblique view of an Anchor Point component pair ready for use.

FIG. 2 is an oblique view of the underside of an Anchor Point component showing multiple release liners.

FIG. 3 is an oblique view of an Anchor Point component with the patch adhered to the skin.

FIG. 4 is an oblique view of a pair of Anchor Point components with the patches adhered to the skin.

FIG. 5 is an oblique view of an Anchor Point connecting a tube to the skin.

FIG. 6a,b,c are a few practices from the prior art.

FIG. 7 is a section view of a tube with an external force applied with the resulting shear forces indicated.

FIG. 8 is a section view of an Anchor Point securing a tube to the skin, with the tube being subjected to normal forces.

FIG. 9 is an oblique view of a box attached to the skin with Anchor Points.

### DETAILED DESCRIPTION OF THE INVENTION

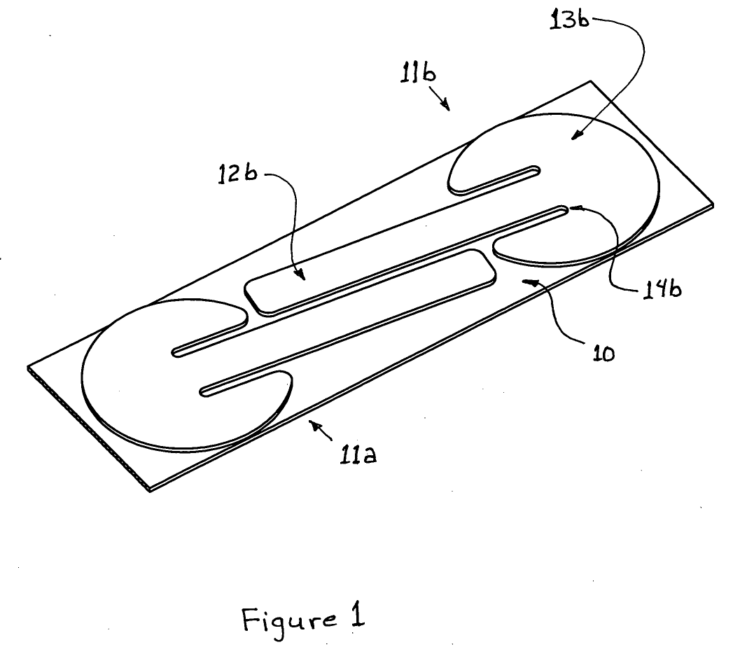

FIG. 1 shows a pair of Anchor Point components 11a and 11b on release liner 10. In this embodiment, each Anchor Point component is composed of a patch section 13 and a tab 12. The patches are shown substantially round but other shapes are within the scope of this invention. The tab interrupts the patch shape so that the tab connects near the center of the patch. The tab is shown with parallel edges. Other tab shapes and end cuts are possible. Full radius relief cuts 14 separate the sides of the tab from the patch.

This embodiment of an Anchor Point is very economical to manufacture. The two Anchor Point components are simply die cut from roll stock supported on a release liner 10. Any size patch and any size tab may be formed with simple steel rule die tooling. The Anchor Point may be made of any convenient material common in use today. Typically, a flexible plastic film is used. Fabric materials are also common in the art. The Anchor Point components are coated with pressure sensitive adhesive. Several suitable adhesives are in common use. One type of tape is Pink Hy-Tape from Hy-Tape Corporation.

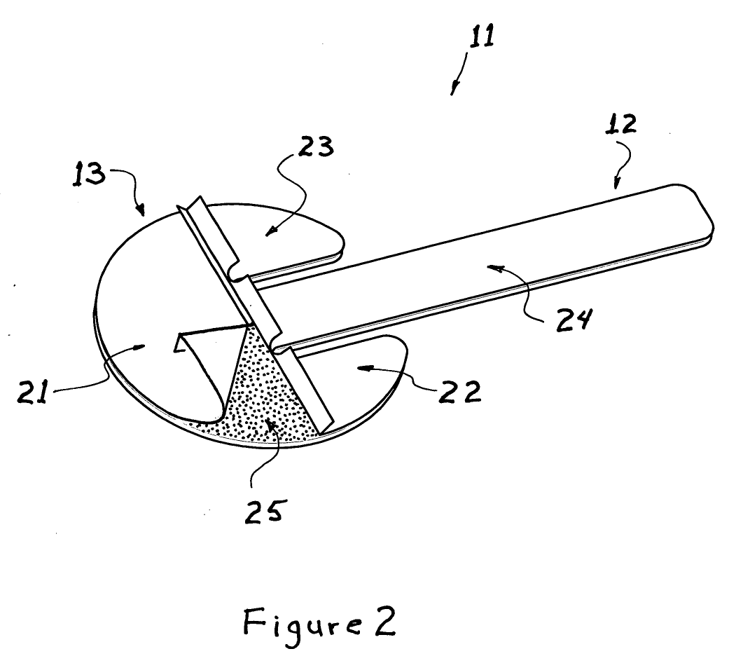

The release liner 10 in FIG. 1 is supplied to protect the adhesive 25 during storage and handling. FIG. 2 shows another embodiment of the release liners. Items 21, 22, 23, and 24 are separate sections of release liner covering the adhesive 25 on component 11. In use, the liner sections 21, 22, and 23 are removed from the patch first, exposing the adhesive on the entire patch. The tab liner 24 is left on the tab until later. In this illustration the liner 21 is peeled back slightly to illustrate the adhesive layer 25. Other liner configurations are feasible to suit the manufacturing process or to facilitate handling or application.

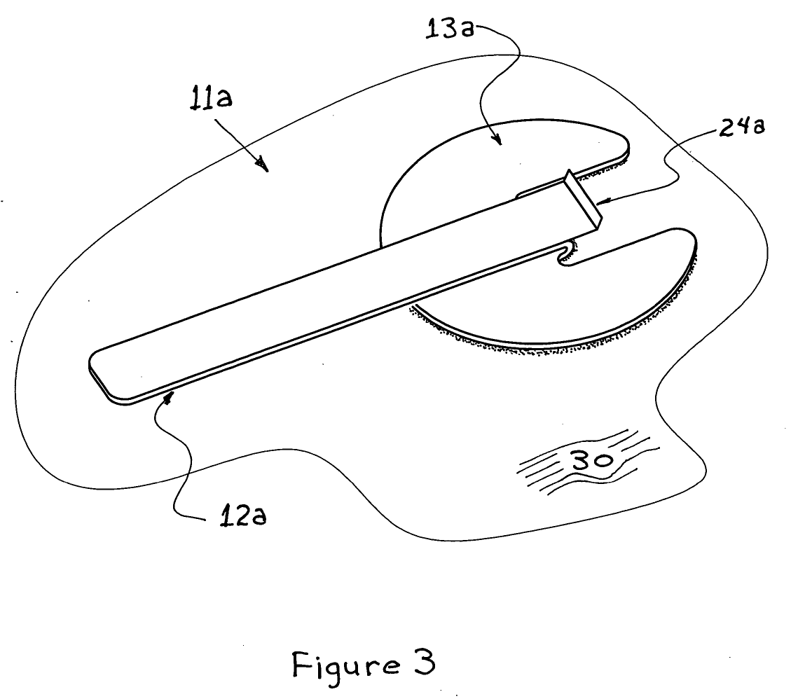

FIG. 3 shows the patch 13a of Anchor Point component Ha attached to the skin 30. The tab 12a is shown folded back over the patch in preparation for application of the second patch. The release liner 24a is still in place. The release liner 24a is not necessary but is convienient for keeping the tab from sticking to the skin. Other release liner configurations will be clear to one skilled in the art.

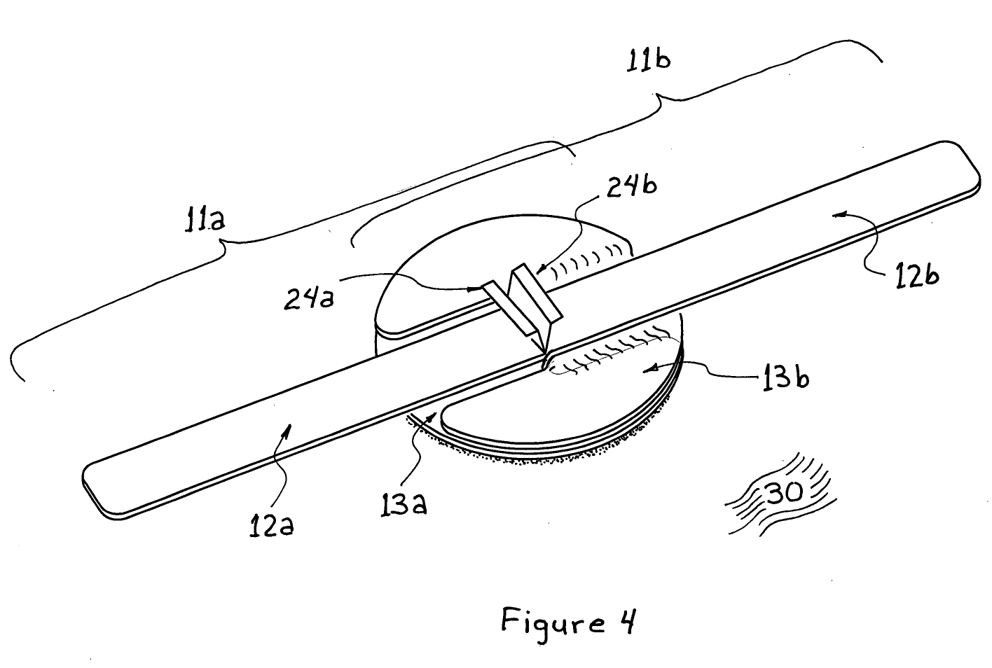

FIG. 4 shows the second Anchor Point component 11b installed over the first component 11a. This arrangement of patches 13a and 13b roughly complete the circle forming a complete circular patch. The pair of tabs 12a and 12b emanate from the center of the patch stack. The release liners 24a and 24b are shown still in place on the tabs. Anchor Points may be pre assembled in this configuration to aid in quick implementation. Pre assembly may be factory supplied or by the practitioner. Factory assemblies may be made by bonding tabs directly to a patch.

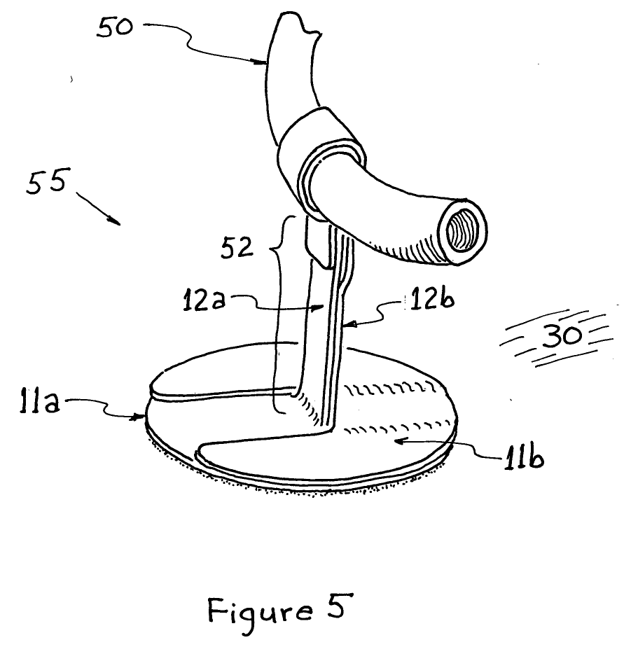

FIG. 5 shows an oblique view of an Anchor Point 55 of assembled components 11a and 11b attaching a tube 50 to the skin 30. The first tab 12a is shown wrapped about the tube and back onto itself. The second tab 12b is shown wrapped around the outside of the first tab. This arrangement leaves no exposed adhesive. The tether length section 52 between the skin and the tube is selected by the user to suit the application. The length may be nil. The tabs may be shortened prior to installation by cutting to length with scissors. Extra tape may be used to extend the tether length as needed.

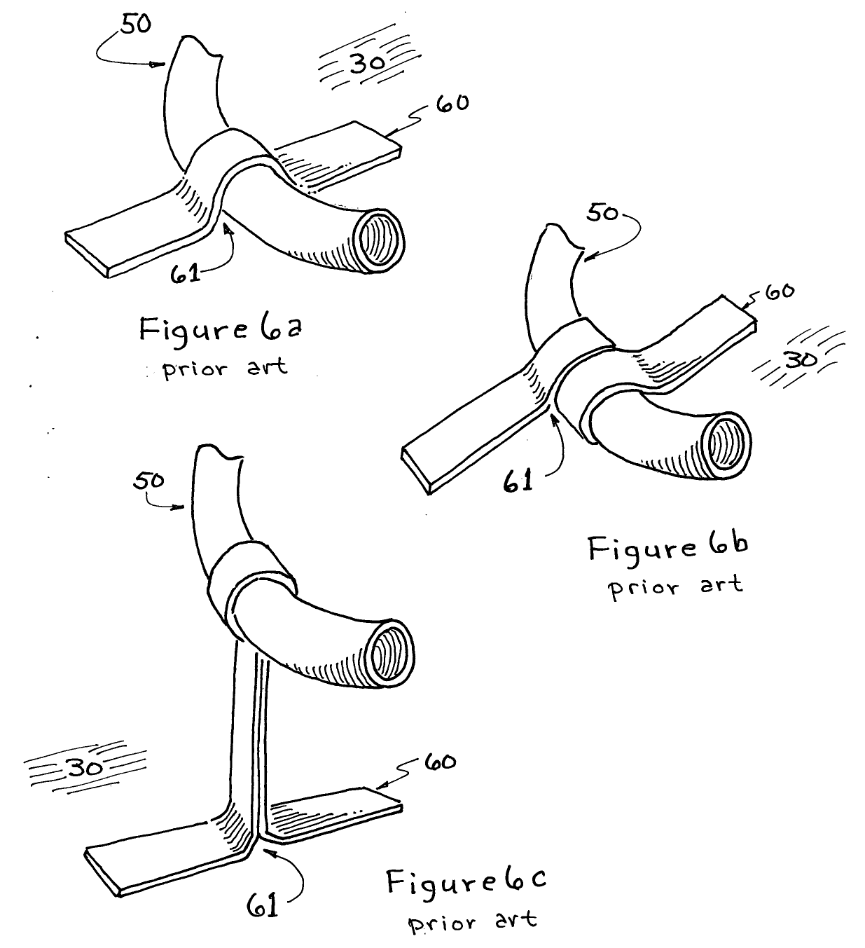

FIGS. 6a, 6b, and 6c show examples from prior art. These are typical of attempts to use a straight section of tape 60 to hold a tube 50 to the skin 30. In each case, a gentle tug on the tube peels the tape away from the skin at 61. Peel occurs when the forces on the adhesive are in the direction perpendicular to the adhesive. Forces directed parallel to the adhesive are said to be in shear. Pressure sensitive adhesives are typically weak in peel and at the same time, strong in shear. The practice shown in FIG. 6c could be improved by the addition of a round patch at the base to prevent peel. This would be yet another embodiment of an Anchor Point.

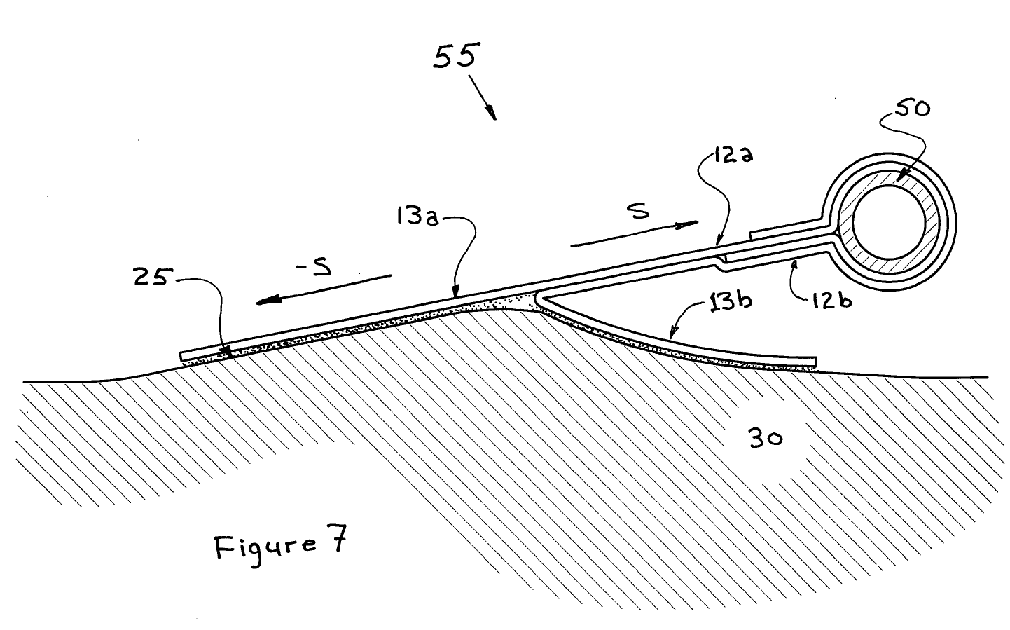

FIG. 7 shows a sectional view of an Anchor Point 55 attached to skin 30 where the tube 50 is being pulled generally in a direction parallel to the skin by a shear force S. For illustrative purposes, the component thickness is shown greatly exaggerated. The skin complies slightly until the force S transferred through the tab 12a to the patch 13a and is balanced by the force of the skin −S on the patch. Under this type of load it is clear that the patch adhesive 25 of 13a is substantially in shear. In this particular direction for S, the patch half on the right shown as 13b is not under load, in shear nor peel.

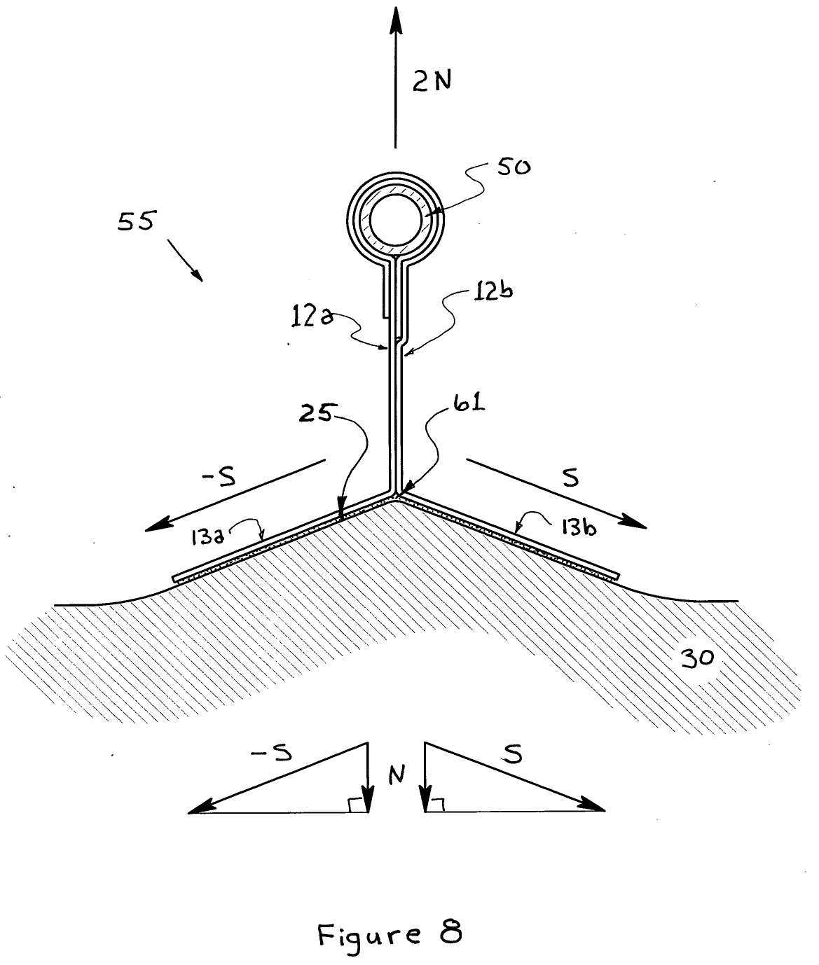

When the force on the tube is directed away from the skin one benefit of the invention becomes clear. FIG. 8 is a section view of an Anchor Point 55 securing a tube 50 to the skin 30. A normal force vector 2N indicates a pulling force on the tube up away from the skin. The tabs would peel up if it were not for the shape and assembly of the patches. Specifically, the full radius relief cuts are closely mated keeping the adhesive from peeling back at point 61. So no peel forces are applied to the patch-skin adhesive 25.

The patches and skin deflect until an equal and opposite force is generated. The skin is lifted until the sum of the components in the normal direction of the shear forces −S and S cancel the normal force. In this example these forces are the same for each side of the patch and so they are both labeled N. In practice, the forces are a combination of FIG. 7 and FIG. 8 and transverse forces, all of which result in shear forces on the patches.

When the Anchor Point needs to be removed or replaced, the patch is easily removed by peeling up the outer edge of the patch from the skin.

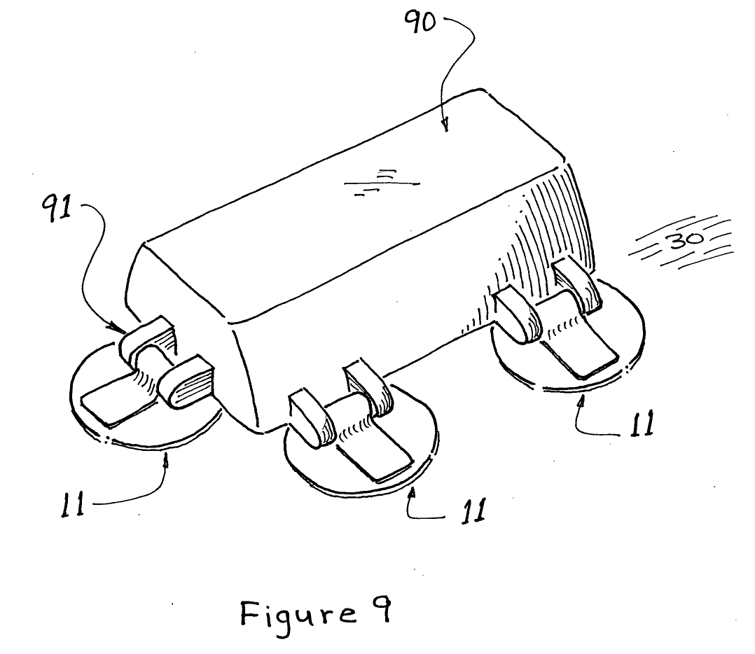

FIG. 9 shows a generic box 90 attached with many Anchor Point components 11. In this application, a single Anchor Point component may be suitable at each attachment point. Although a particular Anchor Point component may be good in shear in some directions, it is not as good in the direction pulling the tab back over the patch. But the other Anchor Point components face other directions. So the weakness of one Anchor Point component is accommodated by the others. The box may be an insulin pump, a data transmitter, a pacemaker, a Holter monitor or other device. This is equally applicable for non medical applications. In this illustration, multiple loops 91 are provided on the box to facilitate attachment. A dual set of Anchor Points may be used to attach to other surfaces by providing patches at each end of the tether section. Anchor Points may be used on other surfaces besides skin. The benefits are more pronounced on flexible surfaces.

The foregoing embodiments are illustrative of the wide range of methodologies and applications of the Anchor Point invention. Other embodiments and applications will be clear to one skilled in the art and are considered to be within the scope of this invention.

What I claim is:

1. An Anchor Point consisting of:
A first tether patch consisting of a substantially round self adhesive patch,
A tether tab emanating from said first patch,
A second tether patch consisting of a substantially round self adhesive patch, and
A tether tab emanating from said second patch.

2. An Anchor Point pair as in claim 1 where the pair is supported on a release liner.

3. An anchor point pair as in claim 1 where the pair is supported on a release liner of several parts including:
A release liner or liners for each patch,
A release liner or liners for each tether tab.

4. An Anchor Point pair as in claim 1 where the pairs are:
Supported on a long strip of release liner material,
Said long strip is scored for removal of individual anchor points.

5. An anchor point pair as in claim 1 where the tabs are substantially emanating from the center of the patch such that forces on the tab are conveyed to the center of the patch resulting in substantially shear forces on the patches.

6. An anchor point pair as in claim 1 sterilized in and protected by a sterilized pouch.

7. A method of applying an anchor point components where:
A self adhesive first patch is applied to a surface,
A self adhesive second patch is applied substantially over the first said patch forming a complete circular patch,
A self adhesive tab emanating from the first patch is wrapped around a tube to be affixed to the surface,
A second self adhesive tab emanating from second said patch is wrapped around said tube.

8. The method described in claim 7 where the tabs convey pulling forces to the center of the patches resulting in substantially shear forces on the patches.

9. The method as described in claim 7 where the patches and tabs are removed from a supporting release liner.

10. The method as described in claim 9 where the patches are removed from a long strip of release liner which is scored to release individual Anchor Point components.

11. A method as described in claim 9 were release liners are removed in sections first from said patches and then from said tabs.

12. An Anchor Point consisting of
A center patch, and
A tab pair emanating substantially from the center of said patch.

13. An Anchor Point as described in claim 12 which is assembled from two flat components that are substantially identical each consisting of:
A patch section, and
A tab section.

14. An Anchor Point as described in claim 12 where the tab pair is bonded substantially to the center of a patch.

15. An Anchor Point as described in claim 12 where forces on the tabs are conveyed to the center of the patch resulting in substantially shear forces on the patch.

16. An Anchor Point as described in claim 12 where the patch is protected by a release liner and the tabs are protected by separate release liners.

17. An Anchor Point consisting of
A single component
Said single component consisting of
A patch section and a tab section.

18. A group of Anchor Points as described in claim 17 where peel forces on one Anchor Point are prevented by shear forces on other Anchor Points.

19. Anchor points as described in claim 17 where components are pre assembled onto a package.

20. An Anchor Point as described in claim 17 where the patch is protected by a release liner and the tab is protected by a separate release liner.
